(19) United States
(12) Patent Application Publication
(10) Pub. No.: US 2026/0335004 A1
(43) Pub. Date: Oct. 29, 2026

(54) Lesion Assessment

(71) Applicant: Sirona Medical Technologies, Inc., Andover, MA (US)

(72) Inventors: Ali Haghighi-Mood, Andover, MA (US); Richard Jonathan Cohen, Chestnut Hill, MA (US)

(21) Appl. No.: 19/655,327

(22) Filed: Apr. 22, 2026

Related U.S. Application Data
(60) Provisional application No. 63/793,437, filed on Apr. 23, 2025.

(51) Int. Cl. A61B 18/14; A61B 18/00; A61B 18/12; A61B 34/00; A61B 34/10

(52) U.S. Cl. CPC A61B 18/1492; A61B 18/1206; A61B 34/10; A61B 34/25; A61B 2018/0016; A61B 2018/00351; A61B 2018/00577; A61B 2018/00642; A61B 2018/00702; A61B 2018/00726; A61B 2018/00761; A61B 2018/00839; A61B 2018/00875; A61B 2018/00904; A61B 2034/102

(57) ABSTRACT

Methods for guiding creation of a durable lesion of desired dimensions in cardiac tissue by application of ablative energy are provided. The methods include receiving first, second, and third impedance data characterizing impedances of patient anatomy as sampled by at least one electrode, determining a distribution and other properties of a field based on the impedance data, a physical structure of the at least one electrode, and parameters of a signal applied to the at least one electrode, determining a boundary of an irreversibly-ablated lesion based on one or more properties of the field and a historical record of a relationship between the field properties and irreversible ablation of tissue, adjusting one or more of the parameters of the signal, a position of the at least one electrode, or force applied to the at least one electrode to alter the boundary of the irreversibly-ablated lesion, and providing the altered boundary.

### CROSS-REFERENCE TO RELATED APPLICATIONS

This application claims priority to U.S. Provisional App. No. 63/793,437, titled “LESION ASSESSMENT” and filed on Apr. 23, 2025, which is hereby incorporated by reference herein in its entirety.

### FIELD

Lesion assessment systems and related methods for use with ablation catheters are provided.

### BACKGROUND

A variety of disorders can negatively affect electrical functioning of the heart, such as arrhythmias that cause too slow a heart rate (bradyarrhythmias) or too rapid a heart rate (tachyarrhythmias). A variety of mechanisms can cause these disorders. For example, abnormal automaticity of the heart's normal pacemaker function can cause disorders of impulse formation cause, and a partial or complete block of electrical impulses can cause disorders of impulse conduction.

One class of treatment involves the ablation of cardiac tissue delivered via a catheter. These catheters can effectively record and/or map electrical impulses in cardiac muscle and can ablate tissue through effective energy application. Treatment of cardiac arrhythmias through use of tissue ablation often requires recording, mapping, and ablating one or more specific target sites within and around a heart of a patient, including on atrial walls and within pulmonary veins. These catheters can be used to ablate target tissue to form lesions that can eliminate or isolate electrical impulses through the ablated tissue, thereby mitigating or correcting the disorder. However, the delivery of energy in this manner can be complicated by various physical realities. Lesion formation at the target site must be irreversible, so precision is paramount. Over-ablation of the tissue can result in unnecessary damage to the patient, while under-ablation can result in ineffective treatment. What's more, blood has a lower impedance than cardiac tissue, so some significant portion of the electrical energy delivered within a patient's heart during the ablation process, even with proper positioning of the catheter and electrode(s), will inevitably pass through the blood and away from the desired treatment zone within tissue. Thus, the total energy delivered by the catheter must compensate for this fact, further complicating lesion formation.

### SUMMARY

Systems and methods for lesion assessment are provided herein.

In an embodiment, a method for guiding creation of a durable lesion of desired dimensions in cardiac tissue by application of ablative energy is provided. The method can include receiving first impedance data characterizing impedance of patient blood as sampled by at least one electrode, receiving second impedance data characterizing impedance of patient tissue as sampled by the at least one electrode, receiving third impedance data characterizing impedance of a target tissue site as sampled by the at least one electrode, determining a field distribution based on the first, second, and third impedance data, a physical structure of the at least one electrode, and parameters of a signal applied to the at least one electrode to deliver an ablative electrical energy, determining a boundary of an irreversibly-ablated lesion based on one or more properties of the field and a historical record of a relationship of one or more properties of the field and the irreversible ablation of tissue, adjusting one or more of the parameters of the signal, a position of the at least one electrode, or a force applied to the at least one electrode to alter the boundary of the irreversibly-ablated lesion, and providing the altered boundary of the irreversibly-ablated lesion.

The method can vary in a number of ways. For example, the at least one electrode can be configured to deliver radiofrequency energy or pulsed field energy. In other example, the field can be an electric field or a current density field. In yet another example, the one or more properties of the field can include at least one of spatial distribution, amplitude, waveform, and duration of the applied field. In still another example, the one or more signal parameters can include an amplitude, a waveform, a duration, and a duty cycle of pulses of the one or more signals. In stilt a further example, the at least one electrode can include a first electrode, a second electrode, and a third electrode, and the first impedance data can be sampled by the first electrode, the second impedance data can be sampled by the second electrode, and the third impedance data can be sampled by the third electrode. In some variations, the patient tissue and the target tissue site can be each located in a patient pulmonary vein. In another example, the method can further include communicating at least one of the one or more signal parameters or boundary of the irreversible lesion to at least one of a signal generator or a mapping system in electronic communication with the at least one electrode. In a further example, the method can include displaying the boundary of the irreversible lesion on a display in electronic communication with the at least one electrode. In still a further example, the method can include determining, based on one or more of the received first, second, third impedance data, and the physical structure of the at least one electrode, a tissue-electrode contact model, and displaying a representation of the tissue-electrode contact model on a display in electronic communication with the at least one electrode. In some variations, the method can further include receiving, based on the tissue-electrode contact model and based on one or more of an adjusted position or adjusted electrode pressure of the one or more electrodes, an indication of an improved degree of contact between the at least one electrode and the target tissue site. In other variations, the method can include receiving first, second, and third electrogram data from the one or more electrodes, wherein the tissue-electrode contact model can include one or more features characterizing the first, second, and third electrogram data. In another example, the method can include assessing tissue-electrode contact or proximity between the at least one electrode and the target tissue site, wherein the third impedance data is based upon the tissue-electrode contact or proximity. In some variations, the method can include assessing the tissue-electrode contact or proximity further comprises obtaining measurements of the tissue-electrode contact via real-time differential impedance monitoring. In another example, the field boundary can be determined by employing a finite element or analytic electrical model of tissue and blood compartments.

In another embodiment, a system is provided. The system can include an ablation catheter having at least one electrode configured to ablate tissue, and a processor in operative communication with the ablation catheter, the processor including a memory storing instructions that, when executed, cause the processor to receive first impedance data characterizing impedance of patient blood as sampled by at least one electrode, receive second impedance data characterizing impedance of patient tissue as sampled by the at least one electrode, receive third impedance data characterizing impedance of a target tissue site as sampled by the at least one electrode, determine a field distribution based on the first, second, and third impedance data, a physical structure of the at least one electrode, and parameters of a signal applied to the at least one electrode to deliver an ablative electrical energy, determine a boundary of an irreversibly-ablated lesion based on one or more properties of the field and a historical record of a relationship of one or more properties of the field and the irreversible ablation of tissue, adjust one or more of the parameters of the signal, a position of the at least one electrode, or a force applied to the at least one electrode to alter the boundary of the irreversibly-ablated lesion, and provide the altered boundary of the irreversibly-ablated lesion.

The system can vary in a number of ways. For example, the one or more properties of the field can include at least one of a spatial distribution, amplitude, waveform and duration of the applied field. In another example, the one or more signal parameters can include an amplitude, a waveform, a duration, and a duty cycle of pulses of the one or more signals. In a further example, the processor can be further configured to communicate at least one of the one or more signal parameters or boundary of the irreversible lesion to at least one of a signal generator or a mapping system in electronic communication with the at least one electrode. In yet another example, the processor can be further configured to display the boundary of the irreversible lesion on a display in electronic communication with the ablation catheter. In a further example, the processor can be further configured to determine, based on one or more of the received first, second, third impedance data, and the physical structure of the at least one electrode, a tissue-electrode contact model, and display a representation of the tissue-electrode contact model on a display in electronic communication with the ablation catheter. In some variations, the processor can be further configured to receive, based on the tissue-electrode contact model and based on one or more of an adjusted position or adjusted electrode pressure of the one or more electrodes, an indication of an improved degree of contact between the at least one electrode and the target tissue site. In another example, the field can be determined by employing a finite element or analytic electrical model of tissue and blood compartments.

In an embodiment, a method for lesion assessment is provided. The method can include receiving anatomy data from one or more electrodes in an electrode array characterizing a measurement of patient anatomy impedance, calibrating a treatment algorithm for the patient based upon the received anatomy data, establishing a zone of irreversible ablation within the patient based upon the treatment algorithm and the one or more contact surfaces, adjusting a strength of signal to the electrode array to alter the zone of irreversible ablation to achieve a desired lesion in the patient tissue, and delivering energy via the electrode array to the patient tissue to create the desired lesion.

The method can vary in a number of ways. For example, the measurement of patient anatomy impedance can include a first measurement of patient blood impedance and a second measurement of patient tissue impedance. In another example, the zone of irreversible ablation can be a function of distance from the one or more electrodes. In yet another example, the method can further include creating a current density model based upon the received anatomy data, wherein establishing the zone of irreversible ablation is further based upon the created current density model. In some variations, the method can further include creating an electric field distribution model based upon the received anatomy data, wherein establishing the zone of irreversible ablation is further based upon the created electric field distribution model. In other variations, creating the current density model can include sub-dividing the one or more electrodes into a plurality of sub-divisions and assessing a contact surface between each sub-division and at least one of patient blood and patient tissue based upon the received anatomy data. In some aspects, sub-dividing the one or more electrodes comprises sub-dividing the one or more electrodes into four equivalent sub-divisions, and wherein assessing the contact surface comprises presupposing one of the four equivalent sub-divisions is in contact with patient tissue and three of the four equivalent sub-divisions is in contact with patient blood. In yet another example, the method can further include assessing a contact surface between the at least one electrode and the patient anatomy, wherein the zone of irreversible ablation is established based upon the contact surface assessment. In some variations, the assessment of the contact surface can include assessing at least one electrode-tissue contact surface and at least one electrode-blood contact surface. In still a further example, the method can include communicating the established boundary of irreversibly ablation to a signal generator, wherein the adjusting a strength of signal to the electrode array further comprises adjusting the strength of signal to the electrode array via the signal generator. In some variations, the method can further include adjusting a position of the one or more electrodes based upon the altered zone of irreversible ablation to achieve a desired lesion position. In another example, the method can include displaying, via at least one display in operative communication with the electrode array, at least one depiction or image of the patient anatomy and a depiction of the zone of irreversible ablation in relation to the at least one image of the patient anatomy. In some variations, the method can further include displaying, via the at least one display, a confidence level for irreversible ablation of patient tissue within the zone of irreversible ablation.

In another embodiment, a system is provided. The system can include an ablation catheter having one or more electrodes configured to ablate tissue, a processor in operative communication with the ablation catheter, the processor including a memory storing instructions that, when executed, cause the processor to receive anatomy data from one or more electrodes in an electrode array characterizing a measurement of patient anatomy impedance, calibrate a treatment algorithm for the patient based upon the received data, establish a zone of irreversible ablation within the patient based upon the treatment algorithm and the one or more contact surfaces; and adjust a strength of signal to the electrode array to alter the zone of irreversible ablation to achieve a desired lesion in the patient tissue.

The system can vary in a number of ways. For example, the measurement of patient anatomy impedance can include a first measurement of patient blood impedance and a second measurement of patient tissue impedance. In another example, the zone of irreversible ablation can be a function of distance from the one or more electrodes. In a further example, the instructions further can cause the processor to create a current density model based upon the received anatomy data, wherein establishing the zone of irreversible ablation is further based upon the created current density model. In some variations, the instructions further can cause the processor to create an electric field distribution model based upon the received anatomy data, wherein establishing the zone of irreversible ablation is further based upon the created electric field distribution model. In other variations, creating the current density model can include sub-dividing the one or more electrodes into a plurality of sub-divisions, and assessing a contact surface between each sub-division and at least one of patient blood and patient tissue based upon the received anatomy data. In some variations, sub-dividing the one or more electrodes can include sub-dividing the one or more electrodes into four equivalent sub-divisions, and wherein assessing the contact surface can include presupposing one of the four equivalent sub-divisions is in contact with patient tissue and three of the four equivalent sub-divisions is in contact with patient blood.

### BRIEF DESCRIPTION OF THE DRAWINGS

The invention will be more fully understood from the following detailed description taken in conjunction with the accompanying drawings, in which:

FIG. 1 is a diagram of a system for cardiac ablation according to some embodiments;

FIG. 2 is a perspective view of an ablation catheter according to an embodiment;

FIG. 3 is a perspective view of an end effector of the ablation catheter of FIG. 2 in a first configuration;

FIG. 4 is perspective view of an end effector of the ablation catheter of FIG. 2 in a second configuration;

FIG. 5 is perspective view of an end effector of the ablation catheter of FIG. 2 in a third configuration;

FIG. 6 is a process diagram of a lesion assessment process according to an embodiment;

FIG. 7 is a process diagram of the lesion assessment process of FIG. 6 with depictions of various sub-steps;

FIG. 8 is a simplified diagram of a sub-divided electrode compatible with the ablation catheter of FIG. 2;

FIG. 9 is a simplified diagram of the sub-divided electrode of FIG. 8 showing a 25% contact electrode-tissue contact area and a 75% electrode-blood contact area;

FIG. 10 is a circuit diagram of a bipolar electrode according to some examples;

FIG. 11 is a simplified diagram of the bipolar electrode circuit of FIG. 10;

FIG. 12 is a simplified diagram of the circuit of FIG. 10 showing tissue and blood impedance;

FIG. 13 is simplified pair of diagram, each of the circuit of FIG. 12, showing relative current flow through blood and tissue;

FIG. 14 is a cross-sectional diagram of each of the bipolar electrodes of the diagram of FIG. 10 according to some examples;

FIG. 15 is a simplified diagram showing a tissue contact assessment procedure according to some examples; and

FIG. 16 is a simplified diagram showing a lesion formation procedure according to some examples.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various systems and methods are provided herein that can assess intracardiac electrode-tissue contact and determine a tissue volume subjected to ablative energy prior to energy delivery. Once determined, software can be used to model the physical properties of the site of interest and then establish and communicate boundaries to the treatment systems. Based upon the model(s) and boundaries, a desired zone of irreversible ablation can be dialed in with much more precision than would be otherwise afforded during such procedures. When dialed in, energy can be delivered to create the lesion(s) necessary to treat the disordered tissue. This precision leads to more effective treatments and better patient outcomes.

To ensure proper placement of the one or more electrodes, mapping techniques have been developed so that the ablation catheter can be guided into position proximate the disordered tissue. By recognizing and pinpointing the unique electrical signature of the tissue and through the use of one or more mapping techniques (e.g., live imaging, anatomical modeling, etc.), a practitioner can maneuver the ablation catheter into position so that its one or more electrodes can deliver their energy to the disordered tissue. Additional details describing mapping techniques can be found in U.S. Patent Publication No. 2023/0293125, entitled, “Cardiac electrical mapping and ablation,” which is incorporated by reference in its entirety. While the described mapping techniques are useful, further steps can be taken to improve precision.

When creating cardiac lesions for treatment of heart rhythm disturbances it is desirable to create irreversible lesions of pre-specified dimensions that permanently block electrical conduction in the tissue so that the heart rhythm disturbance will not recur. Energy sources for ablation of cardiac tissue include radiofrequency energy and pulsed field energy, both of which are electrical in nature. Irreversible tissue ablation occurs when the electric or current density field resulting from the applied ablative energy is sufficiently strong. Regions of tissue where the field is not sufficiently strong may form no lesion or a reversible lesion which later recovers its ability to conduct electrical impulses. The boundaries of the irreversible portion of the cardiac lesion depends on the strength and other properties of the field, which in turn are a function of the structure of the ablation electrode in contact with tissue used to deliver the energy and on the degree of contact of the ablation electrode with the tissue as well as the parameters of the electrical signal applied to the ablation electrode to deliver the ablative energy. If the contact of the ablation electrode is perfect, the current emanating from the electrode will all pass directly into the tissue. If the electrode is only partially in contact with tissue, some of the current will pass directly into the blood pool. The impedance of cardiac tissue is approximately twice that of blood which favors leakage of current into the blood pool. The effectiveness of the applied energy in creating a cardiac lesion is diminished when some of the current emanating from the ablation electrode passes directly into the blood. In particular, the field strength in the tissue is diminished and the dimensions of the boundaries of the irreversible portion of the lesion are reduced, which lead to recurrence of the heart rhythm disturbance.

It is not always feasible for an ablation electrode placed at a target site (e.g., atrial walls and within pulmonary veins) for ablating cardiac tissue to achieve optimal contact with the tissue. Accordingly, in creating a cardiac lesion it is important to able to determine the degree of contact of the ablation electrode with the tissue and determine what effect that this will have on the boundaries of the irreversible portion of the lesion. If the dimensions of the boundaries are not satisfactory, an operator may be able to adjust the dimensions of the boundaries to their desired size by repositioning the ablation electrode or by altering the force applied to the ablation electrode in order to improve electrode-tissue contact or by adjusting the parameters of the electrical signal which applies the ablative energy to the ablation electrode. The signal parameters include the duration, waveform, amplitude, and duty cycle of the pulses that make up the signal and may include other parameters as well.

In an exemplary method, the quality of the contact of the ablation electrode is evaluated by first performing two calibration steps. A first electrode is first placed in the blood pool (no tissue contact) and is used to make a first impedance measurement. A second electrode is place in firm contact with tissue (little or no blood contact) and is used make a second impedance measurement. Then the ablation electrode is placed at the target site for tissue ablation and used to make a third impedance measurement. By comparing the third impedance measurement with the first two impedance measurements one can assess the degree of contact of the ablation electrode with the tissue. Furthermore, based on the known structure of the electrodes and the three measured impedances, a quantitative model is created of the electric field or current density field that is expected to be created as a function of the signal parameters when the ablative energy is applied to the ablation electrode. Based on a previously determined relationship between electric field or current density field properties—including spatial distribution, amplitude, waveform, and the duration for which the field is applied—the expected boundaries of the irreversible portion of the lesion are computed as a function of the signal parameters. The operator may then be able to adjust the dimensions of the boundaries of the irreversible portion of the lesion to their desired size by repositioning the ablation electrode or by altering the force applied to the ablation electrode in order to improve electrode-tissue contact or by adjusting the parameters of the electrical signal which applies the ablative energy to the ablation electrode.

Further, the degree of tissue-electrode contact may be computed and displayed on a graphical user interface and used to guide the operator to adjust the position and pressure applied to the ablation electrode to improve the degree of contact.

Further, the computed boundaries of the region of irreversible ablation may be displayed on a graphical user interface so that the operator may adjust the positioning of the ablation electrode, pressure applied to the ablation electrode, and the signal parameters to optimize the irreversible lesion to be created.

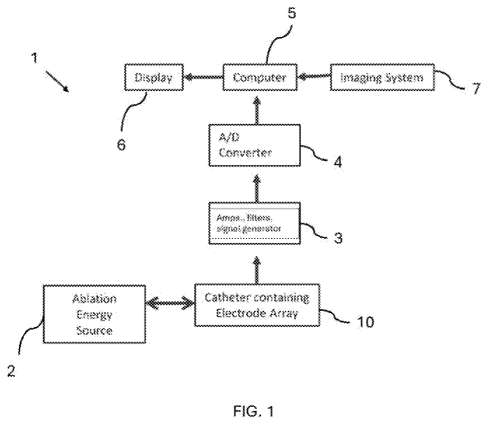

An exemplary system 1, seen in FIG. 1, can involve several components, all in operative communication with each other, including: a catheter 10 containing an electrode array; an ablation energy source 2; various amplifiers and filters, signal generator(s), and other electronic equipment 3 used to adjust and modify system signals (e.g., amplitudes, waveforms, durations, and duty cycles of one or more pulses of one or more signals delivered to the catheter 10 and its electrode array); an Analog-to-Digital Converter (ADC) 4; one or more processors (operating on one or more processors) 5; one or more displays 6; and an imaging system 7. The exemplary system 1 can vary, depending upon the demands of a given procedure, to include additional components, alternative components, or fewer components as required. As described, the system 1, including the described components, can be used to carry out the various calculations and analysis described herein, as well as any variations thereof. The one or more processors can each have at least one memory storing instructions that, when executed by the one or more processors, carry out instructions to receive data, analyze that data, communicate with other components in the system 1, and provide various outputs to a user, the one or more displays 6, etc.

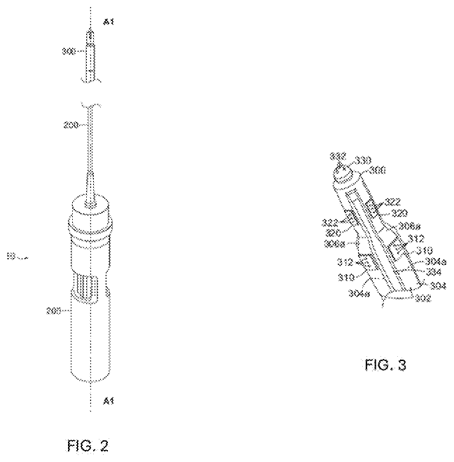

FIGS. 2-5 depict aspects of one exemplary variation of the catheter 10 in greater detail. As depicted, the catheter 10 has a handle 100 on a proximal end, an end effector 300 on a distal end, and an elongate body 200 extending therebetween along a longitudinal axis A1 to connect the handle 100 and the end effector 300.

The end effector 300 is insertable into a body cavity of a patient to be positioned adjacent to and/or in contact with cardiac tissue to record and/or map electrical impulses in the cardiac tissue and to ablate target tissue (e.g., atrial walls and within pulmonary veins), such as during treatment of atrial fibrillation. The illustrated end effector has a plurality of wings 302 that move between at least first, second, and third configurations, a plurality of electrodes 310, 320, one or more irrigation reservoirs 340, 342, a rotational mechanism 350, and a central tube 334 extending along the axis A1. Each wing 302 extends between the distal end of the end effector 300 and the rotational mechanism 350, and each wing has one or more proximal electrodes 310 positioned closer to a proximal end of the end effector 300, one or more mid-electrodes 320 positioned between the proximal electrodes 310 on the wing 302, and flexible irrigation tubes extending therethrough, as discussed in detail below. A distal-most electrode 330 is positioned at the distal end of the end effector 300. Each of the electrodes can deliver ablative energy, including both radiofrequency energy or pulsed field energy, among other kinds.

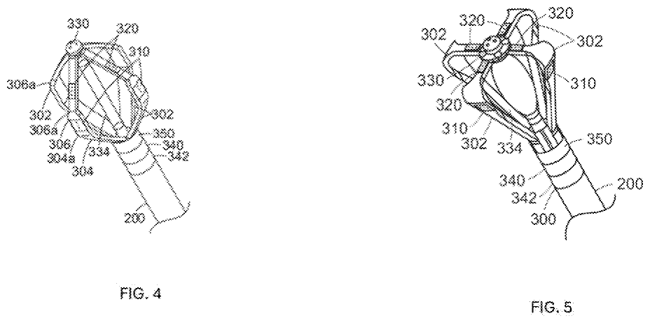

The first, second, and third configurations can be seen respectively in FIGS. 3-5, showcasing the end effector's 300 ability to expand an contract in various ways via its wings 302, which can, in turn, adjust a shape of a lesion formed by the various electrodes 310, 320, 330. The first, linear configuration can be seen in FIG. 3, which can be used, for example, to spot treat tissue. The second, radially expanded configuration can be seen in FIG. 4, which can be used, for example, to treat tissue encircling the end effector 300. The third, distally planar configuration can be seen in FIG. 5, which can be used, for example, to treat a wider surface area of tissue. The wings 302 on the end effector 300 create a flexible structure that can adapt to various shapes to ensure the electrodes can be positioned adjacent to relevant target tissue regardless of an anatomical shape of the target tissue, such as in a pulmonary vein or along an atrial wall. Additionally, the wings 302 can limit any occlusion of blood flow, such as blood flow through a pulmonary vein, to avoid various risks of clot formation and embolization and to avoid difficulties of positioning electrodes experiencing pressure buildup from occluded blood flow. While four wings are illustrated and discussed below, any number of wings can be used, such as between two and ten, and each wing can be made of a variety of different materials, such as plastics, elastomers, metal, various fibers, etc. The wings can have various electrical wires, irrigation tubing or channels, etc. extending therethrough, and the wings can be made of one or more layers of various materials, such as flexible plastics, various polymers, metal and/or metal alloys, etc. Additional details describing end effector variations can be found in U.S. Pat. No. 12,533,182, entitled, “MULTI-MODAL CATHETER FOR IMPROVED ELECTRICAL MAPPING AND ABLATION,” which is incorporated by reference in its entirety.

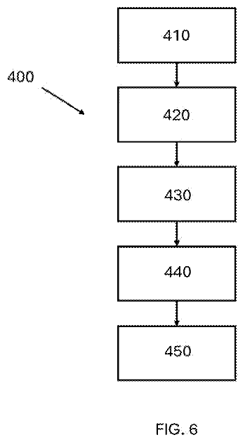

As introduced previously, one challenge faced by practitioners during cardiac ablation procedures is the balance of factors that contribute to the delivery of sufficient energy to the target tissue region in order to properly treat the patient's disorder. These factors include proper placement of the one or more electrodes of the ablation catheter in relation to the afflicted tissue, proper delivery of energy to create a lesion sufficient to treat the afflicted tissue, and mitigation of energy delivery to surrounding bodily structures. FIGS. 6-12 describe exemplary processes, methods, and techniques for practitioners to assess patient anatomy, tune their ablation based upon that anatomy, and then create lesions of desired size and location while minimizing extraneous energy delivery.

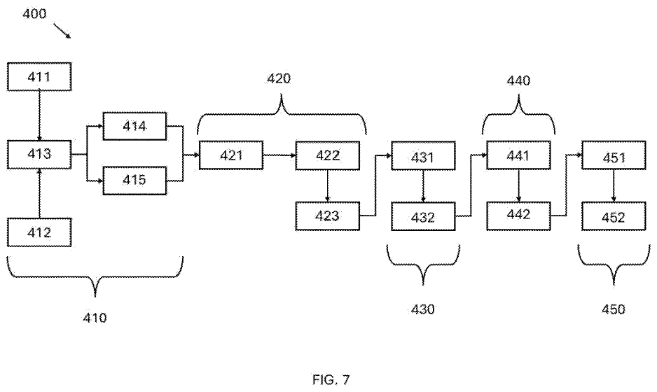

FIG. 6 depicts an example process 400 for lesion assessment using the system 1 according to some variations. Each of the steps in the process 400 may include several sub-steps as described below and as seen with reference to FIG. 7, but these sub-steps of process 400 can be more generally described by larger steps to provide a more general overview for lesion assessment.

Turning now to FIG. 6, the process 400 can begin at step 410, assessment, where data measurements, calibration, and assessment of electrode-anatomy contact surface(s) (or electrode-anatomy proximity if contact is not made), as well as a structure of the electrode(s) themselves inform the system 1 with foundational information necessary to carry out the remaining steps in the process 400. The information received at step 410 can dictate the remaining steps in the process 400 and ultimately the results of the process 400. This information will vary patient-to-patient and based upon electrode position within the patient's anatomy.

At step 420, modelling, the process 400 can create one or more electrical models based upon the information gleaned from step 410 in order to establish a distribution of a field within the patient's anatomy and ultimately establish a boundary of irreversible ablation as a function of distance from the electrode(s) of the catheter 10. For example, the system 1 can determine a distribution of a field within the patient's anatomy based upon information gleaned at step 410, including impedance data, electrode structure, and parameters of a signal applied to the electrode(s). The boundary of irreversible ablation can be established based upon one or more properties of the field as well as a historical record of a relationship between one or more properties of the field and irreversible ablation of tissue.

At step 430, communication, the established boundary of irreversible ablation as a function of distance from step 420 can be communicated out to the rest of system 1 and to the practitioner or other relevant parties. A practitioner or other observer viewing the procedure through various imaging modalities can be informed of the zone of irreversible ablation according to step 420 prior to delivering energy to actually create one or more lesions. For example, where a practitioner is viewing the procedure on a monitor, a digital or virtual overlay surrounding the one or more electrodes of the ablation catheter 10 that are involved in ablation can be presented. This overlay can be a direct presentation of the boundaries established at step 420 and can include a confidence level associated with the boundary, i.e., within the zone, there is a 95% confidence level that irreversible ablation and lesion formation will take place. The overlay can include further boundaries to signal decreased levels of confidence at which irreversible ablation and lesion formation will take place, such as a zone located outside of the 95%-confidence boundary that is associated with a lesser level of confidence at 90%, 85%, or any other level of confidence down to 0% associated with that given distance and the created models of step 420. This additional information can inform the practitioner that they are properly positioned in relation to the tissue to properly treat the patient and minimize the chance of extraneous irreversible ablation to surrounding areas.

At step 440, adjustment, the signal strength and/or electrode placement of the catheter 10 can be adjusted to change the zone of irreversible ablation, such as changing the zone's size and/or shape depending upon the surrounding blood and tissue, as well as depending upon the adjusted electrode-anatomy contact surface(s). This adjustment can be communicated out to the practitioner or other observer in real time via the modalities described with relation to step 430, i.e., through real-time adjustment of the boundary or boundaries and their respective levels of confidence. This zone indicates an area within the patient's anatomy where an irreversibly-ablated lesion would be created should the practitioner deliver energy via the catheter 10 using the present settings.

At step 450, treatment and evaluation, energy can be delivered to the disordered tissue via the ablation catheter and electrodes according to the desired zone of irreversible ablation that was adjusted at step 440, thereby creating one or more lesions in the tissue. When the energy is delivered, lesion formation can be tracked by the system 1 through various modalities, including via medical imaging and the like as described herein in both two- and three-dimensions.

The process 400, as introduced previously, can be broken down further from the stepped-arrangement of FIG. 6 into several sub-steps as seen in FIG. 7. These sub-steps can add further precision to the lesion assessment process, although not every sub-step need necessarily be performed during a given procedure.

For example, step 410, assessment, can be broken down into several sub-steps, labeled sub-steps, 411, 412, 413, 414, 415, which involve the measurement of patient data, calibration of the system 1 based upon that data, and assessing one or more contact surfaces (or general proximity) between the one or more electrodes 310, 320 of the ablation catheter 10 and a patient's anatomy. Each of these sub-steps can contribute information necessary to tailor an ablation procedure to the particular needs of a given patient, and by obtaining patient-specific information, the process 400 can provide effective treatment of cardiac disorders.

The process 400 can start with sub-steps 411, 412, which involve obtaining measurements of a patient's tissue impedance and blood impedance (together “anatomy impedance”). In general, impedance for tissue is about twice that of blood, but patient-specific variations—or even procedure-specific variations—can cause variance in this estimate, hence the need to obtain direct measurement. Impedance values for both blood and tissue can be obtained via the ablation catheter 10 itself through its one or more electrodes 310, 320. A low-amplitude alternating current (AC) can be applied to the blood from the one or more electrodes 310, 320 relied on for sampling, and the resulting voltage can be measured and recorded. To obtain a accurate reading, the one or more electrodes 310, 320 used to sample blood impedance can be fully submerged in blood so that an entire surface area of the one or more electrodes 310, 320 is in blood contact. From there, a blood impedance value can be found. A similar approach can be taken to obtain tissue impedance, which may require direct contact between the one or more sampling electrodes 310, 320 and the tissue of interest. The same or different one or more electrodes 310, 320 used to obtain the blood impedance can be used to obtain tissue impedance. By positioning the one or more electrodes in a position entirely are largely free of blood but in good contact with the tissue to be sampled, a clear measurement of tissue impedance can be obtained. Typically, the sampled tissue will be the tissue similar to that determined to be a source of the disordered cardiac activity so that readjustment of the electrodes 310, 320 following sampling can be minimized, as well as ensuring any differences in impedance values between the sampled tissue and the tissue to be ablated are also minimized. However, if necessary, other tissue can be sampled and used as an estimation for the disordered tissue. While FIG. 7 depicts both of sub-steps 411, 412 taking place, one or both may be omitted from process 400.

By way of example, the impedance values can be measured by the same electrode or my several electrodes any number of times deemed necessary to receive accurate data. To sample blood impedance, a practitioner can place an electrode into a pool of blood, ensuring the electrode is surrounded, and then measuring a first impedance value indicative of the patient's blood impedance. To sample tissue impedance, the practitioner can place an electrode (perhaps the same electrode if desired) firmly in contact with tissue and either entirely or substantially free of blood and measure a second impedance value indicative of the patient's blood impedance. The practitioner can place yet another electrode (which may be an electrode used to sample one or both of the blood and tissue impedances) in contact with tissue at a target site for ablation and use it to measure a third impedance value. This third impedance measurement can also be performed after the calibration of sub-step 413.

At sub-step 413, the system 1 can be calibrated for the patient, and this calibration can be at least partially based on any data gleaned from sub-steps 411, 412. This calibration process can prepare the system for the unique anatomy of the patient undergoing a procedure, but in some variations, this calibration process can be omitted and standard estimates used instead. Where calibration does take place, the system 1 can be tuned specifically for that particular patient. Omission of this calibration may be required where, for example, measurements either cannot be taken or the data received from measurement is insufficient in some way. Alternatively, sub-steps 411, 412, and 413 may roll up together into one calibration sub-step during which impedance measurements or manually-entered data may inform the entirety of the calibration.

At sub-steps 414, 415, one or more contact surfaces can be assessed between the one- or more electrodes 310, 320 of the catheter 10 that are relevant to the treatment of the disordered tissue and the patient's tissue and/or blood. This contact surface assessment may depend on whether impedance values of the tissue and/or blood were measured at sub-steps 411, 412. If one or both measurements were not taken, estimated values may be used instead—such as values characterizing typical tissue or blood impedance—to inform the assessments for sub-steps 414, 415. The one or more contacts surfaces indicate which of the one or more electrodes 310, 320 are in contact with blood or tissue, as well as what proportion of a given electrode 310, 320 is in contact with blood or tissue. In many instances, this assessment may reveal that a given electrode 310, 320 is in contact with both blood and tissue, as well as the proportion of contact between the blood and tissue. By reviewing measured impedance values of the one or more electrodes 310, 320 (as described above with reference to the so-called third impedance value) in comparison to the blood impedance and tissue impedance values obtained at sub-steps 411, 412, surface contact between blood, tissue, and the one or more electrodes 310, 320 being assessed for surface contact can be accurately obtained. This process can be repeated as required for any number of electrodes used in the ablation process. One exemplary technique for determining electrode-tissue contact in particular can rely on the impedance values measured at sub-steps 411, 412 (or stand-in values) and can involve real-time differential impedance monitoring. This monitoring is a high-speed data acquisition technique to extract signal parameters, such as amplitude and/or phase data, in order to assess impedance—and surface contact, in turn—more accurately. Further, the one or more electrodes 310, 320 can be used to receive electrogram data recording electrical activity within the patient's anatomy. The one or more electrodes 310, 320 can monitor changes in electric potential, and this data, like the impedance data, can be taken from a patient's blood, tissue, and target tissue site, such as on location within the pulmonary vein, or elsewhere within the patient's anatomy. This data, too, can be relied on for other aspects of the process 400.

Because the ablation catheter 10 is meant to direct energy to target tissue in order to treat the patient, the assessment of electrode-blood and electrode-tissue contact surfaces may indicate to a practitioner that the current position of the ablation catheter is sub-optimal. The practitioner may, in response to the assessment, need to relocate the ablation catheter in some way for a better contact surface. If this relocation takes place, the process 400 can begin anew or may simply repeat sub-steps 414, 415 to reassess contact surfaces and create an electrode-tissue contact model representing the one or more contact surfaces. A new model can be created as necessary, especially after adjusting movement of or a force applied to the electrodes 310, 320 of the catheter 10, or any other electrode described herein. The electrodes are designed to emit electrical energy outward radially. Because blood impedance is much less than tissue impedance, More electrical energy will flow to the blood than the tissue, so this assessment can be critical. Several physical limitations exist that naturally limit the electrode-tissue contact surface maximum. Generally, cardiac tissue is relatively taut, so an electrode, when placed against cardiac tissue, will never be fully or majorly enveloped by that tissue. Even if some force is applied to the tissue by the electrode, the tissue will not suddenly wrap around the electrode form. Further, blood, as a liquid medium can flow and fill space around an electrode that is not already in contact with tissue. For these reasons, a typical, so-called best-case scenario for cylindrically-shaped electrodes is that about 25% of the relevant surface area of the electrode is in contact with tissue and the remaining 75% in contact with blood. In practice, this can be lower at around 15-20% tissue contact and between 80-85% blood contact. In other examples, such as where the electrodes are flatter in shape with a portion, such as a back side, covered by an insulator, surface contact between tissue and the exposed conducting-portion of the electrode can be higher, even approaching 75% or greater. The processes herein are exemplary and can be generalized and then applied to electrodes of any shape and arrangement.

When step 410, assessment is complete, including any or all of the described sub-steps, the process moves on to step 420, modelling. Step 420 can include different sub-steps 421, 422, which respectively deal with different kinds of electrical models. Sub-step 421 deals with the creation of a current distribution model at the cardiac ablation site and sub-step 422 deals with the creation of an electric field or current density field model at the cardiac ablation site. Each of sub-step 421, 422 is informed by the data gleaned from step 410 sub-steps because the model or models are true to patient anatomy. They are representative of the specific patient reality during the instant procedure. The current distribution model of sub-step 421 represents a model of electrical current per unit surface area of the one or more electrodes 310, 320 as informed by the sampled blood impedance and/or tissue impedance data of sub-steps 411, 412 as well as the surface contact assessments of sub-steps 414, 415. The electric field or current density field model of sub-step 422 represents a similar view of the electric field or current density field of the one or more electrodes 310, 320 as similarly informed by the sampled blood impedance and/or tissue impedance data of sub-steps 411, 412 and the surface contact assessments of sub-steps 414, 415. The distribution of a field within the patient's anatomy can be further determined based upon characteristics of the catheter 10 electrodes themselves, as described in detail below.

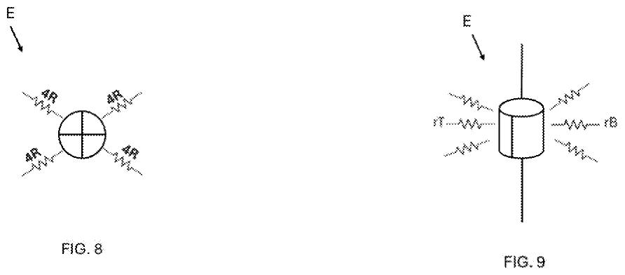

FIGS. 8-14 depict a process by which the system 1 can develop a current distribution model and an electric field or current density field model. The process described assumes the exemplary scenario introduced above for cylindrical electrodes where about 25% of an electrode was measured to contact tissue and the remainder contacts blood. While this specific scenario is described and shown, the process can be generalized and then applied to any given situation as dictated by the actual data, thus creating an electrode contact model. Further, if the degree of contact between a given electrode and patient anatomy is unknown, the general principles described herein can be relied upon to determine degree of contact values based upon data gleaned at step 410 and its associated sub-steps, as well as historical data or estimated data.

The sample electrode E, seen in FIG. 8 and which can be any of the electrodes 310, 320 of the ablation catheter 10, can be sub-divided into n equal segments—here, four segments. Each of the four segments is in parallel, and when exposed to blood, experiences a resistance of value 4R. If 25% of the electrode E is in contact with tissue—equivalent to one of the four segments—and it is found that, when sampled, blood impedance is half of tissue impedance, then the tissue-contact segment experiences a resistance of value 8R. Practically speaking, each of the three blood segments in parallel can be treated as one larger blood-contact segment experiencing a value of 4/3R. FIG. 9 depicts this simplification in which a left-facing surface has some resistance value for the tissue (rT) while a right-facing surface has some resistance value for the blood (rB). This is a simplification and exemplary in nature in order to illustrate the process more generally.

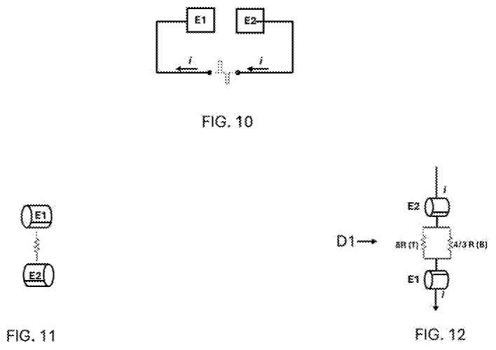

This example will rely on a bipolar (two electrodes) delivery of energy. Where systems rely or require fewer or a greater number of electrodes, such as with the ablation catheter 10 and its electrodes 310, 320, the process can be scaled up as required. In this example, energy can flow between two example electrodes E1, E2 as seen with respect to FIGS. 10-13.

FIG. 10 depicts a general circuit diagram depicting current flow from E2 to E1 in this bipolar example. In this diagram, current i flows from E2 through some portion of the patient P and then to E1. The total current i is conserved. FIG. 11 depicts the general arrangement between E1 and E2 with some impedance R between them. This basic set-up will be relied upon for both determination of the current distribution model of sub-step 421, FIGS. 10-13, and determination of the electric field or current density field model 422, FIGS. 10-11 and 14.

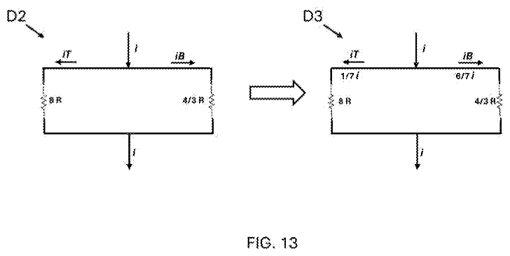

Turning now to FIG. 12, assuming that each electrode E1 and E2 has 25% surface contact with tissue and 75% surface contact with blood, and the impedance of tissue is measured to be twice that of blood, a diagram D1 representing the impedance through tissue and blood together between E1 and E2 can be drawn up. As was the case for FIG. 9, the tissue contact segment experiences an impedance of 8R, and each of the three blood contact segments experiences an impedance of 4R. Together, the three blood contact segments, as segments in parallel, can be treated as one single segment with an impedance value of 4/3R. Thus, the diagram D1 depicts impedance values between electrodes E1, E2 through both tissue and blood according to measured impedance values (or estimated impedance values if measurement is not being relied upon) and according to assess contact surfaces of the electrodes E1, E2. Translating this to current distribution, the diagrams D2 and D3 in FIG. 13 show how the mapped impedance diagram from D1 affects current flow through both blood and tissue. Again, current is conserved, so all current flowing from E2 will flow to E1, and with reference to D2, some fraction of current i will flow through tissue iT and the remaining faction of current i will flow through blood iB. Using Ohm's law or the current divider formula, it can be shown, as seen in D3, that 1/7 of the total current i flows through tissue while 6/7 of the total current i flows through blood. Thus, current distribution between the electrodes E1, E2 can be determined.

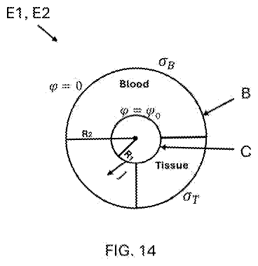

The described determination of a electric field or current density field model is again drawn from the same bipolar electrode set-up explained above. But as stated for the current distribution model, so too can the concepts explained herein be generalized and applied to any ablation catheter, including ablation catheter 10. Turning to FIG. 14 and with reference to FIGS. 10-11, a cross-sectional diagram of an exemplary electrode E1, E2 is provided. The exemplary electrode E1, E2 can act as a stand-in for any of the electrodes described herein, including electrodes 310, 320 and electrodes E1 and E2 as individuals. As shown, the electrode E1, E2 includes a central core C and a surrounding electrode body B. The core C has a radius equal to R₁ and overall electrode E1, E2 has a radius of R₂. Three quarters of the body B are in contact with blood, which has a conductivity of σ_{B}, and one quarter of the body B is in contact with tissue, which has a conductivity of σ_{T}. As shown, the electric potential φ is equal to 0. As also shown, the current density J is depicted by a vector pointing from an outer surface of the core C in a radially outward direction.

In general, several foundations are known to be true. Current density is proportional directly proportional to applied electric field as per Eq. (1) below, where conductivity (σ) is a proportionality constant. This is also known as the vector form of Ohm's law. Applied electric field strength decreases in proportion to distance as per Eq. (2) below, where A is a proportionality constant. Electric potential in this context, is equal to the negative value of the integral of the electric field with respect to distance from R₂ to R₁ (or the positive value of the same integral from R₁ to R₂), which is equal to A multiplied by the natural log of R₂ to R₁ as per Eq. (3). And Eq. (4) solves for constant A.

\vec{J}=σ\vec{E} (1)

E~= (2)

$${φ}_{0} = - {\int }_{R 2}^{R I} E dr = A ln \frac{{R}_{2}}{{R}_{1}} \qquad ( 3 )$$

$$A = \frac{{φ}_{0}}{ln ( {R}_{2} / {R}_{1} )} \qquad ( 4 )$$

Using these foundations, current density can be determined for the electrode E1, E2 of FIG. 14, where in context, as a cylindrical electrode, A is the cross-sectional area of the electrode E1, E2 and L is a length of the electrode E1, E2. Tissue current for the electrode E1, E2 is expressed by Eq. (5), which amounts to tissue contact surface area multiplied by tissue conductivity and divided by distance from the electrode E1, E2. Blood current for the electrode E1, E2 is expressed by Eq. (6), which amounts to blood contact surface area multiplied by blood conductivity and divided by distance from the electrode E1, E2.

$${I}_{T} = \frac{( L ( 2 π ) A / 4 )}{r} {σ}_{T} \qquad ( 5 )$$

$${I}_{B} = \frac{( L ( 2 π ) 3 A / 4 )}{r} {σ}_{B} \qquad ( 6 )$$

When both tissue current and blood current are found, the current fraction flowing through either one is simply the proportion of current flow through that medium as compared to total current. For tissue, the calculation can be seen below per Eq. (7) using the assumption that blood conductivity σ_{B} is half that of tissue conductivity σ_{T}

$${F}_{T} = \frac{{I}_{T}}{{I}_{T} + {I}_{B}} = \frac{( \frac{1}{4} ) {σ}_{T}}{( \frac{1}{4} ) {σ}_{T} + ( \frac{3}{4} ) {σ}_{B}} = \frac{{σ}_{T}}{{σ}_{T} + 3 {σ}_{B}} = \frac{1}{1 + 3 ( \frac{{σ}_{B}}{{σ}_{T}} )} \qquad ( 7 )$$

When reduced, it can be seen that the fraction of current flowing through tissue F_{T} is equal to 1/7 of the total current i. The fraction of current flowing through blood F_{B} is merely the remainder, which is equal to 6/7 of the total current i.

In some variations, steps 410 and 420 may be combined by employing a finite element or analytic electrical conduction model of the tissue and blood compartments in contact with the ablation electrode to compute the electric field or current density field in the surrounding tissue and blood compartments as well as at the surface of the electrode. In this model the electrode itself is treated as a conductor.

From there, the process can proceed to sub-step 423, and a boundary of irreversible ablation can be established as a function of distance or other properties of the determined field distribution in relation to a given electrode as well as parameters of a signal applied to the electrode(s). The boundary can also be determined based upon historical records and prior treatment data of the current or one or more prior patients, including a historical record of a relationship between properties of the field and irreversible ablation of tissue, to increase confidence in measurement values. That is, the boundary represents a zone in which electrical energy delivered by the ablation catheter 10 will create permanent lesions in tissue based upon all of the data and modelling performed thus far. Because the efficacy of ablation drops off as a function of distance, in some variations, the boundary can also include a confidence level indicating likelihood of permanent ablation at a given distance or within a given zone. In some variations, the boundary may be several boundaries located at different distances from one or more electrodes 310, 320, each with a different confidence level for permanent ablation, akin to an elevation map. The actual shape and size of the boundary will depend on the presence and arrangement of the ablation catheter 10 in relation to patient blood and tissue as well as the payload of electrical energy meant to be delivered. Higher energy levels will be more certain to cause permanent ablation and at greater distances, and lower energy levels will be less certain to cause permanent ablation and at lesser distances. The confidence levels can be mapped and assigned according to known irreversible ablation thresholds.

At step 430, communication, the established boundary or boundaries of irreversible ablation (or zone of irreversible ablation, generally) can be communicated out to the rest of the system 1. This communication, for example at sub-step 431, can involve communication with the signal generator 3 and/or to an electro-anatomical mapping system or sub-system, which can be part of the imaging system 7, like those described previously. At sub-step 432, the determined field and/or zone of irreversible ablation can be displayed on one or more displays within the system 1 or in communication with the system 1 so that the practitioner and/or other observers can view the zone in relation to the patient's anatomy. One or more properties of the field, including spatial distribution, amplitude, waveform, and duration, determined previously, can also be displayed. The electro-anatomical mapping module can display the patient's anatomy (or a facsimile of the patient's anatomy) in two- or three-dimensions in real time, i.e., video, a series of images, a virtual representation, etc., and can also depict the ablation catheter 10, including the electrodes 310, 320 in the electrode array, within this anatomical depiction. The zone of irreversible ablation can then be overlayed onto the displayed image, also in two or three dimensions to provide a clear view of the entire procedure site. Where the zone of irreversible ablation comprises multiple boundaries, each boundary can be depicted. Further, where one or more confidence levels for irreversible ablation are calculated, those, too, can be displayed. Additionally, a tissue-electrode contact model, representative of the data described above, can be displayed as well, showing a general proportion of tissue-electrode contact or at least tissue-electrode proximity. If the catheter 10 is moved within the patient, the model can be updated in real-time, which can translate to real-time updates of the display as well. This model and display can assist a practitioner when finding improved tissue-electrode contact for better procedural results. Modeling can be repeated as required to constantly assess tissue-electrode contact in order to maintain accuracy.

Step 440, adjustment, concerns the adjustment of elements of the system 1 to alter the zone of irreversible ablation in real time in accordance with the demands of the procedure. As explained previously, the zone of irreversible ablation is a function of distance from the ablation catheter 10, among other properties. Depending on the payload of electrical energy to be delivered, the zone of irreversible ablation may grow, shrink, or change size and shape. A practitioner may not wish to ablate a large area, instead preferring a more subtle approach, or they may prefer to ablate a much larger area for concern about ineffective treatment. Either way, at sub-step 441, the signal strength transmitted to the electrode catheter 10 via the signal generator can be adjusted to dial in a desired lesion within the tissue based upon the information communicated out in step 430. As the signal strength is adjusted, the zone of irreversible ablation will also be adjusted, and the display version of the zone can be adjusted as well. In general, one or more signal parameters of one or more signals in electronic communication with the catheter 10 and its electrodes 310, 320 can be adjusted to alter the boundary of the zone or boundary of irreversible ablation, and these signal parameters can include an amplitude, a waveform, a duration, and a duty cycle of pulses of one or more signals, as well as any other parameter of the signal(s)

In addition to signal strength adjustment, the practitioner can also adjust the electrode rotation and/or placement in order to achieve the desired lesion or lesions within tissue. In some situations, it may be desirable to achieve overlapping lesions. By adjusting the ablation catheter 10 and electrodes 310, 320 as described, and through the real-time monitoring mentioned previously, the practitioner can see the lesions to be formed upon the delivery of energy. When the practitioner manipulates the catheter, the zone of irreversible ablation and/or model of tissue-electrode contact within the patient can adjust accordingly, moving in relation to the updated electrode contact(s) and positions within the patient. These updates can be passed to any of the displays, such as the display 6 of the system 1, to aid a practitioner during a procedure to find improved contact and positioning to form better lesions.

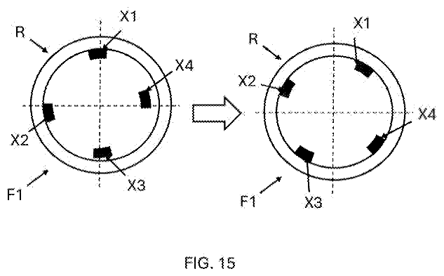

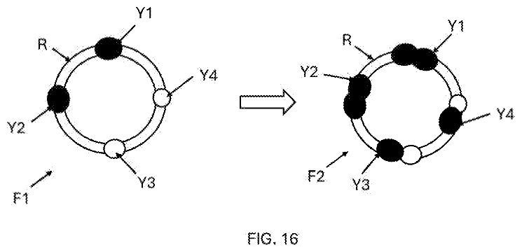

FIGS. 15 and 16 depict a process of catheter rotation for formation of circumferential lesions. As seen in FIG. 15, four exemplary electrodes X1, X2, X3, and X4 are arrayed within a circular tissue region R at a first formation F1 on the left side of FIG. 15. The electrodes X1, X2 are in contact with the region R, electrode X3 is at a first distance from the region R, and electrode X4 is at a second, greater distance from the region R. As ablation takes place, the electrodes X1, X2, X3, and X4 can be rotated relative to the region R to arrive at second formation F2 on a right side of FIG. 15. FIG. 16 depicts the formation of lesions during this process. The first formation F1 results in larger irreversible lesions Y1, Y2, which were created by electrodes X1, X2 being in direct contact with the region R. smaller, irreversible lesions Y3, Y4 were created by electrodes X3, X4 being displaced from the region by some distance. After rotation to the formation F2, each of the lesions Y1, Y2, Y3, Y4 became larger irreversible because of the improved tissue-electrode contacts at electrodes X1, X2, X3, and X4.

At step 450, treatment and evaluation, a practitioner can deliver energy to the tissue to create the desired lesion according to the adjusted zone of irreversible ablation dialed in at step 440. The formation of a lesion is not instantaneous, and it may be desirable to track the lesion as it is formed. Using the features within the system 1, including the various described imaging modalities, mapping techniques, and sampling techniques, a practitioner can track the lesion formation in three dimensions and in real time. This kind of tracking can inform the practitioner on the potential efficacy of the energy delivered via the ablation catheter 10 and can inform them as to whether additional energy delivered to the same or alternative tissue may be required.

In the present disclosure, like-numbered components of the embodiments generally have similar features, and thus within a particular embodiment each feature of each like-numbered component is not necessarily fully elaborated upon. Sizes and shapes of the devices described herein, and the components thereof, can depend at least on the anatomy of the subject in which the devices will be used, the size and shape of components (e.g., spinal rods) with which the devices will be used, and the methods and procedures in which the devices will be used. The figures provided herein are not necessarily to scale. Although the devices and methods disclosed herein are generally directed to surgical techniques, they can also be used in applications outside of the surgical field. Although the invention has been described by reference to specific embodiments, it should be understood that numerous changes may be made within the spirit and scope of the inventive concepts described. Accordingly, it is intended that the invention not be limited to the described embodiments, but that it have the full scope defined by the language of the following claims.

What is claimed is:

1. A method for guiding creation of a durable lesion of desired dimensions in cardiac tissue by application of ablative energy, comprising:
receiving first impedance data characterizing impedance of patient blood as sampled by at least one electrode;
receiving second impedance data characterizing impedance of patient tissue as sampled by the at least one electrode;
receiving third impedance data characterizing impedance of a target tissue site as sampled by the at least one electrode;
determining a distribution of a field based on the first, second, and third impedance data, a physical structure of the at least one electrode, and parameters of a signal applied to the at least one electrode to deliver an ablative electrical energy;
determining a boundary of an irreversibly-ablated lesion based on one or more properties of the field and a historical record of a relationship between one or more properties of the field and irreversible ablation of tissue;
adjusting one or more of the parameters of the signal, a position of the at least one electrode, or a force applied to the at least one electrode to alter the boundary of the irreversibly-ablated lesion; and
providing the altered boundary of the irreversibly-ablated lesion.

2. The method of claim, wherein the at least one electrode is configured to deliver radiofrequency energy or pulsed field energy.

3. The method of claim, wherein the field is an electric field or a current density field.

4. The method of claim, wherein the one or more properties of the field include at least one of spatial distribution, amplitude, waveform, and duration.

5. The method of claim, wherein the one or more parameters of the signal include an amplitude, a waveform, a duration, and a duty cycle of pulses of the one or more signals.

6. The method of claim, wherein the at least one electrode comprises a first electrode, a second electrode, and a third electrode, and wherein the first impedance data is sampled by the first electrode, the second impedance data is sampled by the second electrode, and the third impedance data is sampled by the third electrode.

7. The method of claim, wherein the patient tissue and the target tissue site are each located in a patient pulmonary vein.

8. The method of claim, further comprising communicating at least one of the one or more parameters of the signal or boundary of the irreversible lesion to at least one of a signal generator or a mapping system in electronic communication with the at least one electrode.

9. The method of claim, further comprising displaying the boundary of the irreversible lesion on a display in electronic communication with the at least one electrode.

10. The method of claim, further comprising:
determining, based on one or more of the received first, second, third impedance data, and the physical structure of the at least one electrode, a tissue-electrode contact model; and
displaying a representation of the tissue-electrode contact model on a display in electronic communication with the at least one electrode.

11. The method of claim 10, further comprising repeatedly determining the tissue-electrode contact model after adjusting the position of, or force applied to the one or more electrodes, to indicate an altered degree of contact between the at least one electrode and the target tissue site.

12. The method of claim 10, further comprising receiving first, second, and third electrogram data from the one or more electrodes, wherein the tissue-electrode contact model includes one or more features characterizing the first, second, and third electrogram data.

13. The method of claim, further comprising assessing tissue-electrode contact or proximity between the at least one electrode and the target tissue site.

14. The method of claim 12, wherein assessing the tissue-electrode contact or proximity further comprises obtaining measurements of the tissue-electrode contact via real-time differential impedance monitoring.

15. The method of claim 1, wherein the field is determined by employing a finite element or analytic electrical model of tissue and blood compartments.

16. A system, comprising:
an ablation catheter having at least one electrode configured to ablate tissue;
a processor in operative communication with the ablation catheter, the processor including a memory storing instructions that, when executed, cause the processor to:
receive first impedance data characterizing impedance of patient blood as sampled by at least one electrode;
receive second impedance data characterizing impedance of patient tissue as sampled by the at least one electrode;
receive third impedance data characterizing impedance of a target tissue site as sampled by the at least one electrode;
determine a distribution of a field based on the first, second, and third impedance data, a physical structure of the at least one electrode, and parameters of a signal applied to the at least one electrode to deliver an ablative electrical energy;
determine a boundary of an irreversibly-ablated lesion based on one or more properties of the field and a historical record of a relationship between one or more properties of the field and the irreversible ablation of tissue;
adjust one or more of the parameters of the signal, a position of the at least one electrode, or a force applied to the at least one electrode to alter the boundary of the irreversibly-ablated lesion; and
provide the altered boundary of the irreversibly-ablated lesion.

17. The system of claim, wherein the one or more properties of the field include at least one of a spatial distribution, amplitude, waveform and duration.

18. The system of claim, wherein the one or more parameters of the signal include an amplitude, a waveform, a duration, and a duty cycle of pulses of the one or more signals.

19. The system of claim, wherein the processor is further configured to communicate at least one of the one or more parameters of the signal or boundary of the irreversible lesion to at least one of a signal generator or a mapping system in electronic communication with the at least one electrode.

20. The system of claim, wherein the processor is further configured to display the boundary of the irreversibly-ablated lesion on a display in electronic communication with the ablation catheter.

21. The system of claim, wherein the processor is further configured to:
determine, based on one or more of the received first, second, third impedance data, and the physical structure of the at least one electrode, a tissue-electrode contact model; and
display a representation of the tissue-electrode contact model on a display in electronic communication with the ablation catheter.

22. The system of claim 21, wherein the processor is further configured to repeatedly determine the tissue-contact model after the adjustment of the position of, or force applied to, the one or more electrodes to indicate altered degrees of contact between the at least one electrode and the target tissue site.

23. The system of claim 15, wherein the field is determined by employing a finite element or analytic electrical model of tissue and blood compartments.
